(19) United States
(12) Patent Application Publication
(10) Pub. No.: US 2006/0167355 A1
(43) Pub. Date: Jul. 27, 2006

(54) Method and apparatus for determining peripheral breast thickness

(75) Inventors: Dan Rico, Toronto (CA); Jiwei Yang, North York (CA); Gordon Mawdsley, North York (CA); Martin Yaffe, Toronto (CA); Bindu Augustine, Toronto (CA)

Correspondence Address: BERESKIN AND PARR, 40 KING STREET WEST, BOX 401, TORONTO, ON, M5H 3Y2, CA

(73) Assignee: Sunnybrook and Women's College Health Sciences Centre, Toronto, ON (CA)

(21) Appl. No.: 10/517,601

(22) PCT Filed: Jun. 12, 2003
(86) PCT No.: PCT/CA03/00886

§ 371 Date: Dec. 22, 2005

(30) Foreign Application Priority Data
Jun. 21, 2002 (CA) 2,391,132

(51) Int. Cl. A61B 5/05

(52) U.S. Cl. 600/407

(57) ABSTRACT

A method, computer software product and computer system for analyzing digital mammograms. The method, computer software product and computer system involve a generating phantom thickness object; receiving a set of dimensions for a breast; and, transforming the phantom thickness object to conform to the set of dimensions for the breast to provide the three-dimensional breast thickness object.

### FIELD OF THE INVENTION

This invention relates in general to a method and apparatus for determining the thickness of a breast subjected to a mammogram, and more specifically relates to a method and apparatus for determining the thickness of a breast at its peripheral portion.

### BACKGROUND OF THE INVENTION

In conventional mammography, a woman places her breast on a breast support plate of the mammography machine. A detector is typically mounted under the breast support plate. This detector is sensitive to x-rays. A breast compressor plate that is transparent to light and x-rays presses down against the top of the breast to flatten it and to prevent any movement of the breast during the mammogram. An x-ray source is then turned on to image the breast between the breast support plate and the breast compression plate.

Mammograms provide clues that help to distinguish benign and malignant breast diseases. Radiologists look at both the static appearance of the breast, as well as changes in its structure, micro-classification, density and other characteristics. Breast density determined from the mammogram has been linked to increased link of breast cancer. Women with high mammographic densities (i.e., a high proportion of radiographically-opaque stroma and parenchyma) have been shown to be at an increased risk of breast cancer, when compared to a woman whose breasts are composed mainly of fatty or adipose tissue. Classification of radiological appearance of mammograms on the basis of the general distribution of parenchyma, stroma and fat, can yield very strong estimates of breast cancer risk.

In the mammography field, various systems and methods have been developed to quantify breast density in terms of the fraction of the projected breast area that is occupied by radiographically dense tissue. These methods suffer from at least two limitations. First, they do not use information about three-dimensional conformation of the breast. A simple area measurement may provide an erroneous measure of the actual amount of fibroglandular tissue in the breast.

The computation of volumetric density in a compressed breast is based on both image data and knowledge of the thickness at each pixel. However, at the breast periphery, where the breast is not bounded by either the breast support plate or the breast compression plate, the thickness of the breast may not be known. However, this thickness is required to determine volumetric density of the compressed breast.

Accordingly, a method and apparatus for determining the thickness of a breast at its periphery is desirable.

### SUMMARY OF THE INVENTION

An object of one aspect of the present invention is to provide a method of generating a three-dimensional breast thickness object for a digital mammogram of a breast.

In accordance with one aspect of the present invention, there is provided a method of generating a three-dimensional breast thickness object for a digital mammogram of a breast. The method comprises:

(a) generating a phantom thickness object for transforming into the breast thickness object, the phantom thickness object being generated in a three-dimensional modeling means and being substantially breast-shaped;

(b) determining a set of dimensions for the breast; and

(c) transforming the phantom thickness object to conform to the set of dimensions to provide the three-dimensional breast thickness object in the three-dimensional modeling means.

An object of a second aspect of the present invention is to provide a computer program product for use on a computer system for analyzing digital mammograms.

In accordance with a second aspect of the present invention, there is provided a computer program product for use on a computer system or analyzing digital mammograms. The computer program product comprises:

(a) a recording medium;

(b) phantom thickness object generation means recorded on the recording medium for instructing the computer system to generate the phantom thickness object;

(c) data entry generation means recorded on the recording medium for instructing the computer system to upload a set of dimensions for the breast; and,

(d) transformation generation means recorded on the recording medium for instructing the computer system to transform the phantom thickness object to conform to the set of dimensions for the breast to provide the three-dimensional breast thickness object

An object of a third aspect of the present invention is to provide a computer system for analyzing digital mammograms.

In accordance with a third aspect of the present invention, there is provided a computer system for analyzing digital mammograms. The computer system comprises:

(a) phantom thickness object generation means for generating the phantom thickness object;

(b) data entry means for receiving a set of dimensions for a breast; and,

(c) transformation means for transforming the phantom thickness object to conform to the set of dimensions for the breast to provide the three-dimensional breast thickness object.

### BRIEF DESCRIPTION OF THE DRAWINGS

A detailed description of preferred aspects of the invention is provided herein below with reference to the following drawings, in which:

FIG. 1, in a perspective view, illustrates a mammography machine;

FIG. 2, in a sectional view, illustrates a breast phantom image constructed of poly-methyl-methacrylate (PMMA);

FIG. 3, in a perspective view, illustrates a three-dimensional triangle phantom image;

FIG. 4, in a schematic view, illustrates a breast compressed during a mammogram;

FIG. 5 is a graph of polynomial conversion functions obtained from the three-dimensional triangle phantom image of FIG. 3;

FIG. 6 is a graph of a grey value histogram of a digital mammogram;

FIG. 7 shows a phantom thickness map object;

FIG. 8 shows a digital mammogram object;

FIG. 9 illustrates the phantom thickness map object of FIG. 7 including internal and external sets of landmarks;

FIG. 10 illustrates the digital mammogram object of FIG. 8 including internal and external sets of landmarks;

FIG. 11 is a graph of a thickness profile for the phantom thickness map object of FIG. 7;

FIG. 12 is a graph illustrating a thickness profile of the digital mammogram of FIG. 8;

FIG. 13 is a flowchart illustrating a method of generating a phantom thickness map in accordance with an aspect of the invention;

FIG. 14 is a flowchart illustrating a method of generating a breast thickness object in accordance with a preferred aspect of the invention;

FIG. 15 is a flowchart illustrating a method of generating phantom landmarks for the phantom thickness map object of FIG. 13; and,

FIG. 16 is a flowchart illustrating a method of determining breast landmarks of the breast thickness object in accordance with an aspect of the invention.

### DETAILED DESCRIPTION OF PREFERRED EMBODIMENTS OF THE INVENTION

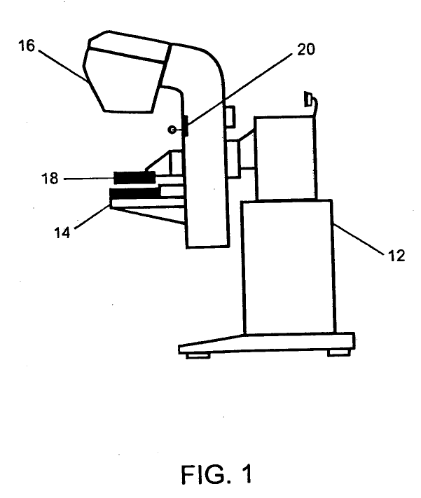

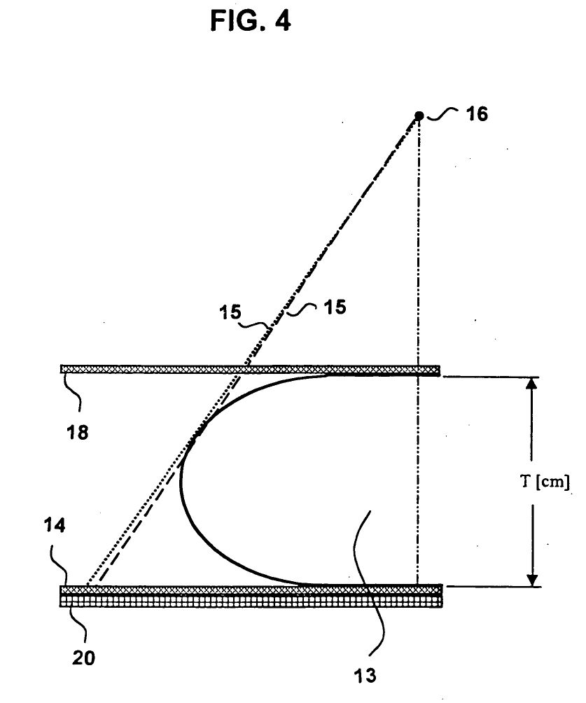

Referring to FIG. 1, there is illustrated in a perspective view, a mammography machine 12. The mammography machine 12 includes a breast support plate 14, a breast compression plate 18, and an x-ray tube 16. In operation, the x-ray tube 16 projects x-rays through the breast compression plate 18, which is transparent to light and x-rays, through the breast, and through the breast support plate 14. The breast compression plate 18 may be vertically adjusted to accommodate breasts of different dimensions. The breast support plate 14 includes a detector (shown in FIG. 4) that is sensitive to the x-rays. Variation in the density and thickness of the breast will affect the x-rays traveling through the breast. This in turn will affect the image left on the detector in the breast support plate 14. These signal variations may then be examined for possible malignancies or other conditions. However, to determine density, and thus to properly interpret the image, the thickness of the breast must be known at all points.

Referring to FIG. 4, a breast that is compressed between the breast support plate 14 and the breast compression plate 18 is shown in a schematic view. The breast 13 is of a thickness T in centimeters. X-rays originating from an x-ray tube 16 project through the breast compression plate 18, any empty space surrounding the breast, the breast, and breast support plate 14 to impinge on a detector 20 underneath the breast support plate. When they impinge on the detector 20, the x-rays 15 contain information about the thickness and composition of that portion of the breast through which they have passed. However, the x-rays will also have been affected by the empty space between the breast support plate 14 and breast compression plate 18 through which they have passed. At some points, of course, where the breast is in contact with both the breast support plate 14 and breast compression plate 18, there will be no empty space. However, at other points, the curvature of the breast creates a space between the breast compression plate 18 and the breast support plate 14 that is not occupied by the breast. If the thickness of the breast is known, then this information can be taken into account when interpreting the x-ray data on the detector 20.

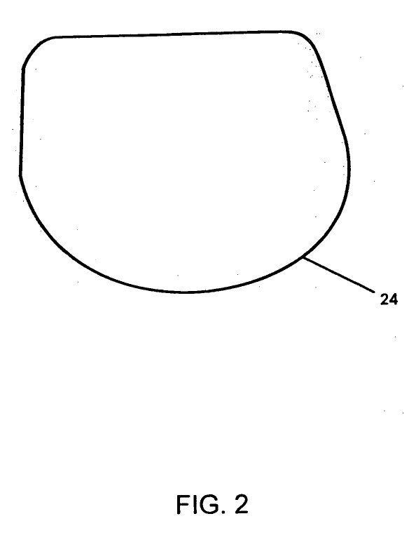

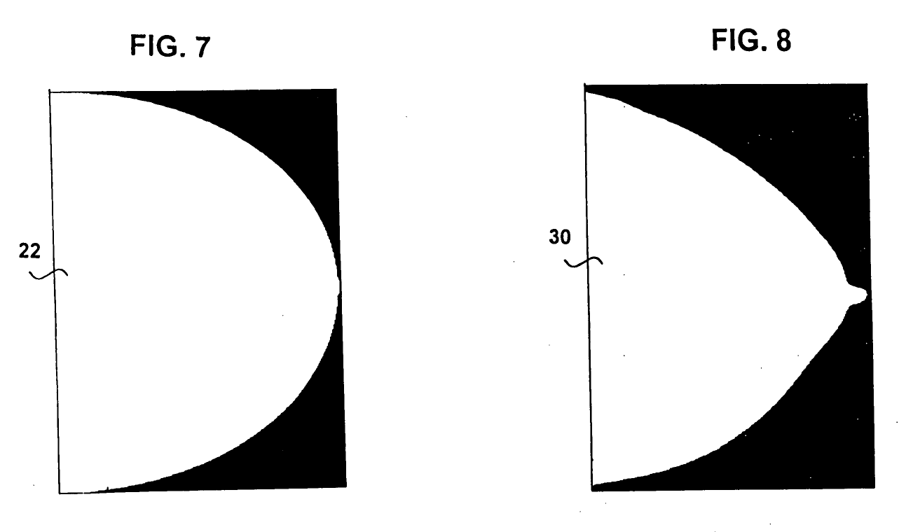

Referring to FIG. 7, there is illustrated a phantom thickness map object 22 generated using three-dimensional modeling software in accordance with an aspect of the present invention. This phantom thickness map object 22 is generated using a breast phantom 24 constructed of poly-methyl-methacrylate (PMMA) shown in FIG. 2. This phantom breast 24 is first imaged by the mammography machine 12 to obtain a phantom mammogram. As the composition of the phantom mammogram image is uniform and known, the intensity of the x-rays transmitted through the phantom breast 24 will vary based on the variation in the thickness of the phantom breast 24.

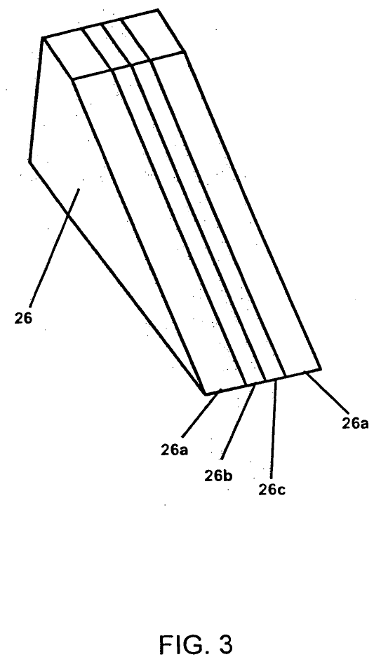

Referring to FIG. 3, there is illustrated in a perspective view, a three-dimensional triangular phantom 26 in accordance with an aspect of the invention. This triangular phantom 26 contains slabs of PMMA 26a, as well as plastic layers 26b and 26c simulating 30% and 50% fibroglandular tissue respectively (from left to right—PMMA, 30%, 50%, PMMA). This three-dimensional triangular phantom 26 is then subjected to a mammogram to generate a set of image data. Again, this image data will vary only with the thickness of the triangular phantom 26. However, the thickness of the triangular phantom 26 will be known at any point. Thus, the set of image data for the triangular phantom 26 can be used to correlate the thicknesses of the triangular phantom 26, with particular points in the phantom mammogram having the same intensity of x-ray transmitted, and therefore being of the same thickness.

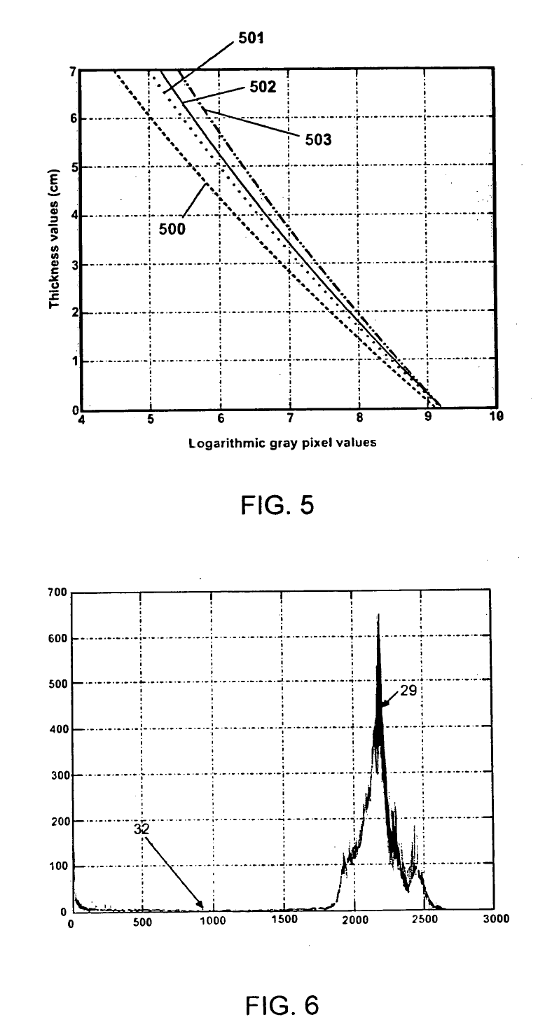

From the x-ray profile along the PMMA triangular phantom image from top seven centimeters to base less than one millimeter, the position along the wedge (i.e. the thickness) is determined from the logarithm of the image pixel signal by interpolation using a second-degree polynomial fit. This fit is plotted as line 501 on the graph of FIG. 5. The polynomial function represented by line 501 of FIG. 5 allows direct conversion from logarithmic gray pixel value to thickness value.

In a similar way, second-degree polynomial functions are found for 30% fibroglandular tissue and for 50% fibroglandular tissue. The second-degree polynomial function for 30% fibroglandular tissue is plotted as line 503 in FIG. 5, and the second-degree polynomial function for 50% fibroglandular tissue is plotted as line 502 in FIG. 5. A second-degree polynomial function for 100% fibroglandular tissue was obtained by mirroring the 30% polynomial function around the 50% polynomial function, and is represented as line 500 in FIG. 5. Any percentage glandular composition can be verified by using slabs of known thickness and composition.

The phantom thickness map 22 and polynomial functions 500, 501, 502 and 503 can then be used to compute the thickness and density map of a particular digital mammogram. First, this will require the phantom thickness map 22 to be rescaled to the size of the digital mammogram, and will require the thickness values of the phantom thickness map 22 to be normalized to the thickness readout of the mammographic system. Next, the phantom thickness map is overlaid on a digital mammogram image using a point-based elastic warping method, which is efficient at recovering local deformations (see F. Bookstein, Thin-Plate Splines and the Decomposition of Deformations, IEEE Transactions Pattern Analysis and Machine Intelligence, 11, pp. 567-585, 1989). With this technique, special care is needed in the selection of landmarks. Two different sets of landmarks are chosen, both in the phantom thickness map 22 and in the digital mammogram.

The phantom thickness object of FIG. 7 is defined and determined as the object with thickness values larger than zero. Referring to FIG. 6, there is illustrated an intensity histogram 29 of a digital mammogram. This intensity histogram is bimodal. The breast thickness object 30 of FIG. 8 is automatically generated from the histogram using a threshold value 32 shown in FIG. 6. This threshold value 32 is at the middle point of the valley between the two modes in the histogram. The boundaries of both the phantom thickness object of FIG. 7 and the breast thickness object of FIG. 8 are found by employing a morphological removing operation. In the binary images of FIG. 7 and FIG. 8, a pixel is set to zero (black) if all of its four-connected neighbours are one (white), thus leaving only the boundary pixels on.

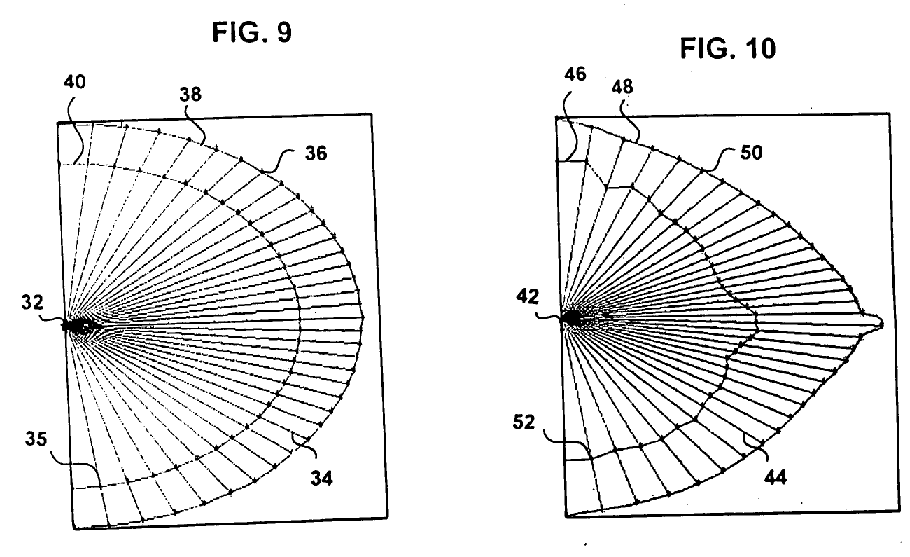

Having generated the phantom thickness object 22 of FIG. 7, and the breast thickness object 30 of FIG. 8, it is possible to select landmarks. Referring to FIGS. 8 and 9 respectively, the phantom thickness object 22 and breast thickness object 30 are shown divided into segments. In the case of the phantom thickness object 22 of FIG. 9, these segments are defined by a series of radial lines 34 extending from the center 32 of the phantom thickness object 22 to the outer edge of the phantom thickness object 22. Each of these radial lines intersects a first phantom boundary line 38 marking the outer edge of the phantom thickness object 22. Together, these intersection points provide a first set of phantom landmarks 36. Similarly, in the case of the breast thickness object 30 of FIG. 10, the segments are defined by a series of radial lines 44 extending from the center 42 of the breast thickness object 30 to the outer edge of the breast thickness object 30. Each of these radial lines intersects a first breast boundary line 48 marking the outer edge of the breast thickness object 30. Together, these intersection points provide a first set of breast landmarks 50.

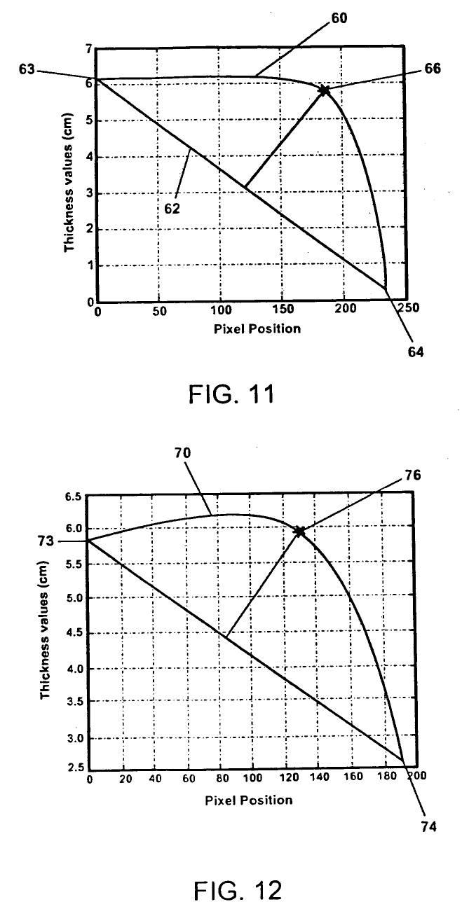

Referring to FIG. 9, a second phantom boundary line inside the first phantom boundary line is shown. This boundary line represents the point at which the breast phantom 24 is no longer in contact with the breast compression plate 18. This point is selected from a phantom thickness profile 60 of FIG. 11. Each of the radial lines 34 of FIG. 9 has an associated thickness profile such as the thickness profile 60 of FIG. 11. A line 62 is drawn connecting the first point 63 and last point 64 of the thickness profile 60. A point 66 on the thickness profile 60 is then selected to be a maximum distance from the line 62. This point 66 is substantially at the point where the radial line 34 of the phantom ceases being in contact with the breast compression plate 18. Together, the points 66 selected for all of the radial lines 34, generate the second boundary line 40.

A breast thickness profile 70 is plotted for each of the radial lines 44 of the segmented breast thickness object 30 of FIG. 10. The logarithmic profile values are converted to thickness using the polynomial function for 50% dense material. A thickness profile 70 of one such radial line 44 is shown in FIG. 12. Unlike the thickness profile 60 of the breast phantom 24, the thickness of an actual breast is not uniform over a first interval, but instead increases before decreasing. Similar to the case of FIG. 11, a line 72 connecting the first point 73 on the profile 70 with the last point 74 on the profile 70 is drawn. Then, a point 76 is selected to be a maximum distance from the line 72. These points 76 for all of the radial lines 44 are then plotted as points 52 on the segmented breast thickness object 30 of FIG. 10, and are connected to provide the second breast boundary line 46. Unlike the second phantom boundary line 40 of the phantom object of FIG. 9, the second boundary line 46 of the breast thickness object of FIG. 10 is irregular, reflecting variation in the composition and compressibility of the breast.

The minimum thickness values for thickness on the outer edge of the breast thickness object are computed using the polynomial function for 100% dense material, to convert logarithmical grey pixel values to thickness. The polynomial function for 100% dense material is selected due to the layer of skin surrounding the breast. A corrected warped thickness map is then computed by cropping the radial lines 44 and cropping the map generally, at the minimum thickness value given by the 100% conversion function. Next, the cropped profile is approximated by a linear combination of two exponentials using a non-linear least squares logarithm.

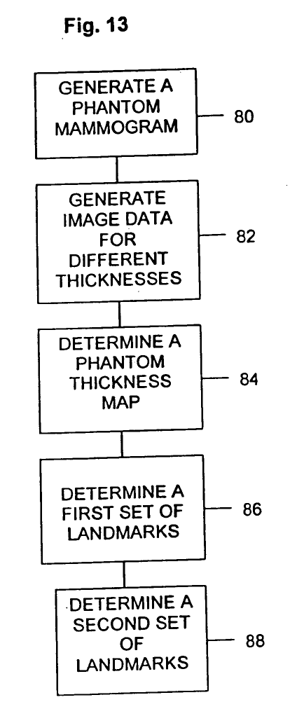

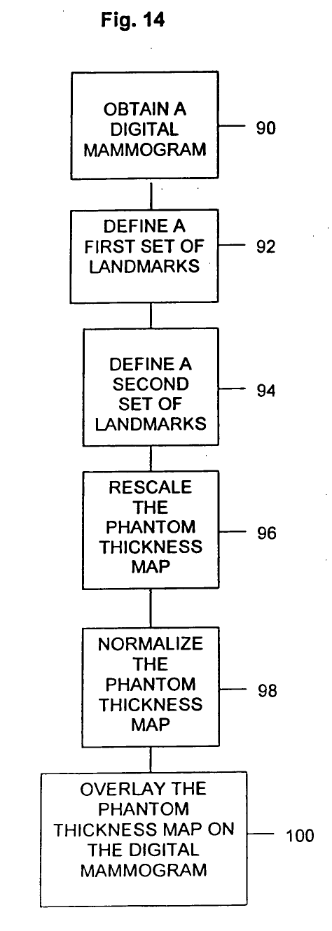

Referring to FIGS. 13 and 14, there is illustrated in flowcharts a method of generating a breast thickness map in accordance with an aspect of the present invention. In step 80 of the flowchart of FIG. 13, a phantom mammogram is obtained by imaging a breast phantom 24. This phantom mammogram contains a series of profiles of the breast phantom along different planes, reflecting the difference in thickness of the breast phantom at these different planes. The image data for different thicknesses is then generated by imaging a three-dimensional triangular phantom 26. This triangular phantom contains slabs of PMMA, as well as plastics simulating 30 and 50% of fibroglandular tissue. By imaging this three-dimensional triangular phantom 26, image data for known and different thicknesses are generated. This information can then be combined with the information provided by step 80, to determine a phantom thickness map object 22 in step 84. Then, in steps 86 and 88, a first set of phantom landmarks, and a second set of phantom landmarks are determined.

Referring to FIG. 14, a digital mammogram of an actual breast is obtained in step 90. Then, in steps 92 and 94 respectively, a first set of breast landmarks, and a second set of breast landmarks are defined. In step 96, the phantom thickness map is rescaled to the size of the digital mammogram, and in step 98, the phantom thickness map object is normalized by normalizing its thickness size to the thickness readout of the mammography system. In step 100, the phantom thickness map object is overlaid on the digital mammogram using a point-based elastic warping method, which is efficient at recovering local deformations.

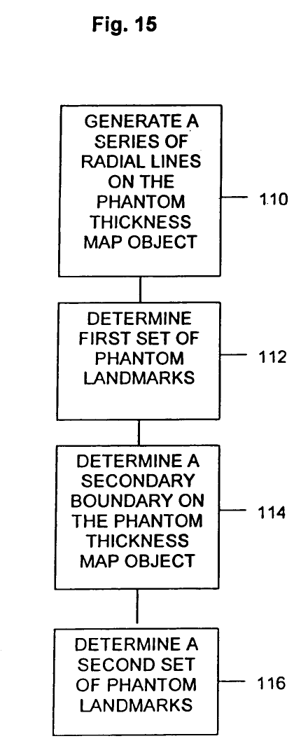

Referring to FIG. 15, there is illustrated in a flowchart a method of selecting a first set of phantom landmarks and a second set of phantom landmarks in accordance with an aspect of the present invention. In step 110, a series of radial lines extending from the center of the phantom thickness object 22 to its outer edge are generated. In step 112, a first set of phantom landmarks are determined by taking the intersection of these radial lines with the outer edge of the phantom thickness object. Then, in step 114, a secondary boundary of the phantom thickness object 22 is determined. This secondary boundary of the phantom thickness object is defined by the points at which the phantom thickness object moves from being in contact with the breast compression plate, to not being in contact with the breast compression plate. Then, in step 116, a second set of phantom landmarks is determined. This second set of phantom landmarks is determined by taking the intersection of the radial lines generated in step 110 with the secondary boundary generated in step 114.

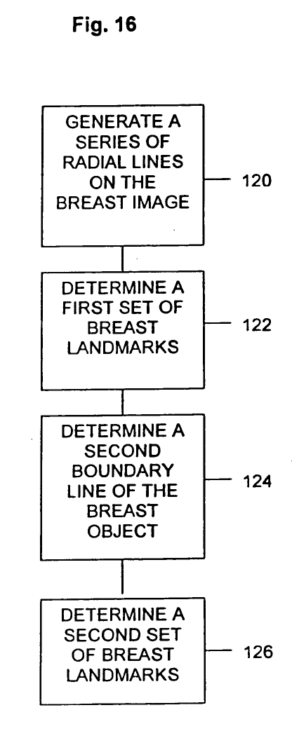

Referring to FIG. 16, there is illustrated in a flowchart a method of selecting a first set of breast landmarks and a second set of breast landmarks in accordance with an aspect of the present invention. In step 120, a series of radial lines extending from the center of the breast image to its outer edge are generated. In step 122, a first set of breast landmarks are determined by taking the intersection of these radial lines with the outer edge of the breast image. Then, in step 124, a secondary boundary of the breast image is determined. This secondary boundary of the breast image is defined by the points at which the breast changes from being in contact with the breast compression plate, to not being in contact with the breast compression plate. Then, in step 126, a second set of breast landmarks is determined. This second set of breast landmarks is determined by taking the intersection of the radial lines generated in step 120 with the secondary boundary generated in step 124.

According to a preferred aspect of the present invention, step 100 of the flowchart of FIG. 14 is executed by applying the point-based elastic warping method to warp the first set of phantom landmarks into the first set of breast landmarks, and to warp the second set of phantom landmarks into the second set of breast landmarks.

Other variations and modifications of the invention are possible. For example, phantom thickness objects may be generated in other ways by, say, for example, assembling an average breast from a series of mammograms for different women, or by selecting a stored breast thickness object that most closely matches the shape and dimensions of the breast being imaged from a library of previously obtained breast thickness objects. Further, other techniques may be applied to overlay the phantom thickness map on the breast thickness object. All such modifications or variations are believed to be within the sphere and scope of the invention as defined by the claims appended hereto.

1. A method of generating a three-dimensional breast thickness object for a digital mammogram of a breast, the method comprising:
(a) generating a phantom thickness object for transforming into the breast thickness object, the phantom thickness object being generated in a three-dimensional modeling means and being substantially breast-shaped;
(b) determining a set of dimensions for the breast; and
(c) transforming the phantom thickness object to conform to the set of dimensions to provide the three-dimensional breast thickness object in the three-dimensional modeling means.

2. The method as defined in claim 1 wherein the set of dimensions comprises a thickness readout for the breast and a size of the digital mammogram and wherein step (c) comprises
normalizing a set of thickness values of the phantom thickness object based on the thickness readout for the breast; and,
rescaling the phantom thickness object to the size of the digital mammogram.

3. The method as defined in claim 2 further comprising
determining a set of phantom landmarks at the edge of the phantom thickness object;
determining a set of breast landmarks at the edge of the digital mammogram; and
warping the phantom thickness object to map the set of phantom landmarks onto the set of breast landmarks.

4. The method as defined in claim 3 further comprising
determining a second set of phantom landmarks on the phantom thickness object;
estimating a breast density at a second set of points in the digital mammogram to determine a breast local thickness at the second set of point and a second set of breast landmarks corresponding to the second set of points; and
warping the phantom thickness object to map the second set, of phantom landmarks onto the second set of breast landmarks.

5. A computer program product for use on a computer system for analyzing digital mammograms, the computer program product comprising
(a) a recording medium;
(b) phantom thickness object generation means recorded on the recording medium for instructing the computer system to generate the phantom thickness object;
(c) data entry generation means recorded on the recording medium for instructing the computer system to upload a set of dimensions for the breast; and,
(d) transformation generation means recorded on the recording medium for instructing the computer system to transform the phantom thickness object to conform to the set of dimensions for the breast to provide the three-dimensional breast thickness object.

6. The computer program product as defined in claim 5 wherein the set of dimensions comprises a thickness readout for the breast and a size of the digital mammogram, and wherein the transformation generation means comprises
normalizing means for instructing the computer system to normalize a set of thickness values of the phantom thickness object based on the thickness readout of the breast;
rescaling means for instructing the computer system to rescale the phantom thickness object to the size of the digital mammogram.

7. The computer program product as defined in claim 6 further comprising
first phantom landmark generation means recorded on the recording medium for instructing the computer system to determine a set of phantom landmarks at the edge of the phantom thickness object; and
first breast landmark generation means recorded on the recording medium for instructing the computer system to determine a set of breast landmarks at the edge of the digital mammogram;
wherein the transformation generation means is operable to instruct the computer system to warp the phantom thickness object to map the set of phantom landmarks onto the set of breast landmarks.

8. The computer program product as defined in claim 7 further comprising
second phantom landmark generation means recorded on the recording medium for instructing the computer system to determine a second set of phantom landmarks at the edge of the phantom thickness object; and
second breast landmark generation means recorded on the recording medium for instructing the computer system to estimate a breast density at a second set of points in the digital mammogram to determine a breast local thickness at the second set of point and a second set of breast landmarks corresponding to the second set of points;
wherein the transformation generation means is operable to instruct the computer system to warp the phantom thickness object to map the second set of phantom landmarks onto the second set of breast landmarks.

9. A computer system for analyzing digital mammograms, the computer system comprising
(a) phantom thickness object generation means for generating the phantom thickness object;
(b) data entry means for receiving a set of dimensions for a breast; and,
(c) transformation means for transforming the phantom thickness object to conform to the set of dimensions for the breast to provide the three-dimensional breast thickness object.

10. The computer system as defined in claim 9 wherein the set of dimensions comprises a thickness readout for the breast and a size of the digital mammogram, and wherein the transformation means comprises
normalizing means for normalizing a set of thickness values of the phantom thickness object based on the thickness readout of the breast; and,
rescaling means for resealing the phantom thickness object to the size of the digital mammogram.

11. The computer system as defined in claim 10 further comprising
first phantom landmark determining means for determining a set of phantom landmarks at the edge of the phantom thickness object; and
first breast landmark determining means for determining a set of breast landmarks at the edge of the digital mammogram;
wherein the transformation means is operable to warp the phantom thickness object to map the set of phantom landmarks onto the set of breast landmarks.

12. The computer system as defined in claim 11 further comprising
second phantom landmark determining means for determining a second set of phantom landmarks at the edge of the phantom thickness object; and
second breast landmark generation determining means for estimating a breast density at a second set of points in the digital mammogram to determine a breast local thickness at the second set of point and a second set of breast landmarks corresponding to the second set of points;
wherein the transformation means is operable to warp the phantom thickness object to map the second set of phantom landmarks onto the second set of breast landmarks.
